(19) United States
(12) Patent Application Publication
(10) Pub. No.: US 2002/0098213 A1
(43) Pub. Date: Jul. 25, 2002

(54) USE OF ELLAGIC ACID AND ITS DERIVATIVES IN COSMETICS AND DERMATOLOGY

(76) Inventors: FREDERIC BONTE, ORLEANS (FR); ALEX SAUNOIS, ORLEANS (FR)

Correspondence Address: DENNISON, SCHULTZ & DOUGHERTY, 1745 JEFFERSON DAVIS HIGHWAY, ARLINGTON, VA, 22202, US

(21) Appl. No.: 09/508,670

(22) PCT Filed: Oct. 1, 1998
(86) PCT No.: PCT/FR98/02098

(30) Foreign Application Priority Data
Oct. 1, 1997 (FR) 97 12227

(51) Int. Cl. A61K 6/00; A61K 7/00

(52) U.S. Cl. 424/401

(57) ABSTRACT

The invention relates to the use of ellagic acid and its derivatives in the field of cosmetics and pharmacy, especially dermatology.

It relates more particularly to all applications where it is desired to reinforce the dermal-epidermal junction or improve hair condition by increasing the proportion of collagen VII in the presence of keratinocytes and/or fibroblasts.

In particular, these applications involve toning up the skin, reducing wrinkles or improving hair condition.

The present invention relates essentially to a novel use of ellagic acid, its salts, its metal complexes or its mono- or polyether or mono- or polyacylated derivatives in the field of cosmetics or pharmacy, especially dermatology.

Ellagic acid, which also has the name 2,3,7,8-tetrahydroxy(1)-benzopyrano-pyrano (5,4,3-cde)(1)benzopyran-5,10-dione, is a well-known molecule belonging to the group of the polyphenols and is found in the plant kingdom. Reference may be made to the publication Merck Index 20th edition (1996), no. 3588.

Document FR-A-1 478 523 has disclosed a process for the purification of ellagic acid and the purified ellagic acids obtained by such a process.

Ellagic acid has the following chemical formula:

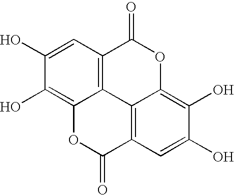

which contains four fused rings.

Ellagic acid is commercially available in particular from Extra Synthèse, France.

Document EP-A-0 496 173 describes extracts of Aleppo galls containing ellagic acid in combination with gallic acid and hydrolyzable tannins, and an application in cosmetics as an ultraviolet B protection filter and in a preventive role against the harmful effects of free radicals, which are responsible for skin ageing.

It is further known that type VII collagen, hereafter referred to as collagen VII, is the predominant constituent of the anchoring fibrils associated with the basal membrane joining the epidermis to the dermis. It is synthesized by the keratinocytes of the basal layer of the epidermis and to a lesser extent by the fibroblasts of the dermis, as described by R. Burgeson in the publication entitled “Type VII collagen, anchoring fibrils, and epidermolysis bullosa”, J. Invest. Dermatol. (1993) 101, 252-255. Reference may also be made to the publication by A. Koenig et al. in J. Invest. Dermatol. (1992) 99 808-812. It will be noted in this connection that, according to recent studies, topical applications of retinoic acid increase the number of anchoring fibrils on skin which has undergone actinic ageing (Woodley D. T. et al., J. Amer. Med. Assoc. (1990) 263, 3057-9). Now, retinoic acid, or tretinoin, is recognized as being one of the most effective antiwrinkle agents (L. H. Kligman, Cutis (1988) 41 (6) 419-20; J. J. Leyden et al., J. Am. Acad. Dermatol. (1989) 21 (3Pt 2) 638-44; J. H. Saurat, Horm. Res. (1995) 43 (1-3) 89-92).

According to the publication by Y. Q. Chen, A. Mauviel, J. Ryynanen, S. Sollberg, J. Uitto (“Type VII collagen gene expression by human skin fibroblasts and keratinocytes in culture: Influence of donor age and cytokine responses”, J. Invest. Dermatol. (1994) 102, 205-209), certain manifestations of skin ageing, such as increased delicacy of the skin and reduced ability of the epidermis to repair itself, might be attributable to a decrease in the synthesis of collagen VII in elderly subjects. It will be noted that the expression “delicacy of the skin” particularly covers the appearance of blisters at the sub-basal level.

In J. Invest. Dermatol. (1995) 105 844-850, M. Akiyama et al. described that collagen, particularly collagen VII, played an important role at the level of the human hair follicle, especially at the level of the basal membranes of the matrix (peripapillary zone) and at the level of the basal membrane of the bulge (at the top of the bulb). These two zones contain cells of high mitotic potential, particularly keratinocytes which produce the hair shaft.

Finally, it is also known that the dermis-epidermis cohesion is of prime importance for the basal populations of the epidermal keratinocytes to have an optimum metabolism, a good cohesion thus enabling them in particular to assure the formation of a good-quality, elastic and well-formed corneal layer with optimum internal hydration which respects the functionalities of the cellular layers. A good dermis-epidermis cohesion thus participates in the formation and maintenance of skin at metabolic equilibrium, giving it especially a good esthetic appearance.

Within the framework of the present invention, it was surprisingly discovered that ellagic acid, its salts, its metal complexes or its mono- or polyether or mono- or polyacylated derivatives afforded a significant increase in the proportion of collagen VII in a medium in which normal human keratinocytes were present.

This finding led to the development of a novel cosmetic, pharmaceutical or especially dermatological composition useful more particularly in all applications where it is desired to increase the proportion of collagen VII, particularly with a view on the one hand to favoring the cohesion between the dermis and the epidermis, and on the other hand, at the level of the hair follicles of the scalp, to contributing towards the restoration or maintenance of the functionality of the cells, particularly the keratinocytes. This property proved particularly useful for the preparation of topical, cosmetic or dermatological compositions.

Such compositions make it possible in particular to favor the cohesion between the dermis and the epidermis in persons whose skin is atonic or loose. They can also be useful in hair care for improving hair condition, promoting the growth of good-quality hair or slowing down or delaying hair loss. They also make it possible to treat pathological conditions accompanied by a deficiency of the dermal-epidermal junction, such as epidermolysis bullosa.

Thus, according to a first feature, the invention relates to the use of ellagic acid, its salts, its metal complexes or its mono- or polyether or mono- or poly-acylated derivatives as a cosmetic agent for improving the cohesion between the dermis and the epidermis, said agent preferably being incorporated into a cosmetic composition comprising a cosmetically acceptable vehicle.

Advantageously, the improvement in the cohesion between the dermis and the epidermis is realized by reinforcing the dermal-epidermal junction.

According to a second feature, the present invention also covers the use of ellagic acid, its salts, its metal complexes or its mono- or polyether or mono- or polyacylated derivatives as a cosmetic agent for increasing the proportion of collagen VII, said agent preferably being incorporated into a cosmetic composition comprising a cosmetically acceptable vehicle.

Within the framework of the invention, it has in fact been clearly demonstrated that, surprisingly, the action of ellagic acid, its salts, its metal complexes or its mono- or polyether or mono- or polyacylated derivatives is to increase the proportion of collagen VII.

Thus the compositions of the invention prove particularly useful in all applications where it is desired to improve the cohesion between the dermis and the epidermis.

Within the framework of the invention, the ellagic acid salts include particularly metal salts, especially those of alkali metals or alkaline earth metals such as sodium and calcium, amine salts such as those of methylglutamine, diethanolamine, triethanolamine, choline and bis-triethylamine, and amino acid salts, especially those of basic amino acids such as arginine, lysine and ornithine, the metal complexes include particularly those with zinc and copper, and the mono- or polyacylated derivatives comprise particularly saturated or unsaturated acyl groups having from 2 to 22 carbon atoms. These acyl groups preferably correspond to acetic, palmitic, oleic, linoleic, linolenic, arachidonic, stearic, brassidic, erucic, behenic and (all Z)-5,8,11,14,17-eicosapentaenoic acids. The above-mentioned mono- or polyether derivatives are particularly derivatives of alkoxy comprising from 1 to 4 carbon atoms, or condensation derivatives of one or more of the hydroxyl groups of ellagic acid with a sugar or a chain of sugars. In particular, said derivatives are 3-methoxyellagic acid or mono- or polyether derivatives with sugars such as glucose, arabinose, rhamnose and galactose.

The above-mentioned ether or acylated derivatives can be obtained by polyphenol etherification or acylation processes well known to those skilled in the art. Some of them can also be obtained by extraction from plants.

In addition, the ellagic acid, its metal complex salts or its mono- or polyether or mono- or polyacylated derivatives will be particularly intended for toning up the skin, preventing or delaying the appearance of signs of skin ageing, delaying the appearance of wrinkles or reducing their depth, and improving hair condition.

The invention thus relates in particular to antiwrinkle products, products for combating skin ageing, particularly natural skin ageing, and loosening of the skin, or of a hair care lotion such as a lotion for combating hair loss.

Consequently, the invention makes it possible to prepare particularly valuable cosmetic compositions for combating skin ageing, particularly actinic ageing of the skin, i.e. ageing induced by radiation, particularly solar radiation and very particularly ultraviolet solar radiation.

In general terms, the cosmetic compositions of the invention prove particularly useful as skin toning products, particularly for combating loose or atonic skin.

According to a third feature, the invention further relates to the use of ellagic acid, its salts, its metal complexes or its mono- or polyether or mono- or polyacylated derivatives for the preparation of a pharmaceutical composition, especially dermatological composition, for treating pathological conditions associated with a deficiency in the cohesion between the dermis and the epidermis, particularly conditions associated with a weakening of the dermal-epidermal junction, such as epidermolysis bullosa, or for treating manifestations or pathological conditions associated with an insufficiency of collagen VII.

The cosmetic or pharmaceutical compositions, especially dermatological compositions, of the invention will advantageously contain from 0.0001% to 5% by weight, preferably between 0.01 and 1% by weight, of ellagic acid, its salts, its metal complexes or its mono- or polyether or mono- or polyacylated derivatives, based on the total weight of the composition.

As a further advantage, the compositions of the invention may comprise at least one substance which favors the synthesis of the constituents of the extracellular matrix of the skin and/or regulates the formation of a good-quality corneal layer.

Examples of such substances which may be mentioned are vitamins, particularly vitamins of groups A and C, and their derivatives such as esters, tocopherols, xanthines, particularly caffeine or theophylline, retinoids, particularly vitamin A acid, extracts of Centella asiatica, asiatic and madecassic acids and their glycosylated derivatives such as asiaticoside or madecassoside, extracts of Siegesbeckia orientalis, extracts of Commiphora mukul and extracts of Eriobotrya japonica, and mineral elements, preferably magnesium, manganese, silicon and zinc, these mineral elements advantageously being used in the form of the chloride in the case of magnesium and manganese, in the form of silanol in the case of silicon and in the form of aspartate in the case of magnesium.

Furthermore, the composition according to the invention can also contain at least one substance selected from the group consisting of aliphatic C₃-C₁₂ alpha-hydroxy acids, particularly citric acid, malic acid and lactic acid, amino acids, particularly arginine, citrulline and threonine, ceramides, glycoceramides, phospholipids, slimming agents, forskolin, extracts of Coleus, extracts of Tephrosia, agents for combating stretch marks, particularly extracts of horse-chestnut and escin, agents for protecting or improving the microcirculation, particularly the bioflavonoids of Ginkgo biloba, and sun filters, particularly titanium oxides, acyl methoxycinnamate (Parsol MCX®) and filters of vegetable origin.

In the compositions of the invention which are intended more particularly for hair treatment and care, the ellagic acid, its salts, its metal complexes or its mono- or polyether or mono- or polyacylated derivatives will advantageously be associated with at least one other active principle selected from the group consisting of antidandruff agents such as extracts of Arctium lappa, chloroxylenol, resorcinol or zinc pyrithione, antiseborrhea agents such as a 5α-reductase inhibitor, particularly an extract of Pygeum africanum, and agents for stimulating the blood microcirculation, such as cepharanthine and methyl nicotinate.

The formulations can take a variety of forms. One of the most widely used forms is a topical form suitable for application to the skin tissue. Without implying a limitation, these appropriate topical formulations include emulsions, creams, milks, balms, gels, lotions and medicated make-up compositions.

According to other features, the invention further relates to a method of cosmetic or pharmaceutical treatment, especially dermatological treatment, aimed at increasing the proportion of collagen VII.

The invention further relates in particular to a method of cosmetic treatment for improving the cohesion between the dermis and the epidermis, particularly by reinforcing the dermal-epidermal junction, for toning up the skin, for preventing or delaying the appearance of signs of skin ageing, for delaying the appearance of wrinkles or reducing their depth, and for improving hair condition, characterized in that it consists in delivering a cosmetically effective amount of ellagic acid, its salts, its metal complexes or its mono- or polyether or mono- or polyacylated derivatives, optionally contained in a cosmetically acceptable excipient.

Embodiments of this method, in particular, naturally result from the foregoing description relating to the compositions.

Other objects, characteristics and advantages of the invention will become clearly apparent to those skilled in the art from the following explanatory description relating to several Examples, which are given simply by way of illustration and cannot in any way limit the scope of the invention. The Examples form an integral part of the invention. Also, in the Examples below, the percentages are indicated by weight, the temperatures are in degrees Celsius and the pressure is atmospheric pressure, unless indicated otherwise.

### EXAMPLES OF THE INVENTION

### Example 1

### Demonstration of the Activity of Ellagic Acid in Increasing the Proportion of Collagen VII in a Culture of Normal Human Keratinocytes

The test below was carried out on ellagic acid available from Extra Synthèse, France, in the form of a powder melting above 360° C.

The tests were carried out blind on normal human keratinocytes.

### 1°Test protocol

### a) Origin of the Keratinocytes

The cultures of normal human keratinocytes (NHK) are prepared from a surgical sample of healthy skin. In the present study, the tests were carried out on two strains of cells originating from face-lifts performed on a 49-year-old caucasian woman, denoted by NHK 1124, and a 50-year-old caucasian woman, denoted by NHK 1106.

### b) Culture Conditions

The keratinocytes are kept in complete serum free medium (denoted by SFMc, GIBCO). The cells were subcultured once or twice from the primary culture (i.e. one pass, denoted by P1, or two passes, denoted by P2) before being used for the test.

### c) Treatment Conditions

The cells are inoculated in a 96-well culture plate at a rate of 25,000 NHK per well in SFMc. After incubation for 24 h, which is necessary for good adhesion of the cells, the medium is replaced with SFMc diluted to 2% to limit the proliferation of the keratinocytes during the test. The test concentration of ellagic acid is 0.5 microgram per ml of test medium, which is obtained immediately beforehand from a stock solution containing 0.5 mg of ellagic acid per ml of DMSO. The final concentration of DMSO in the test medium is therefore 0.1% V/V_{final}. This concentration is not toxic to the cells. The control consists of SFMc test medium diluted to 2%, to which 0.1% V/V of DMSO has been added. Six cultures are prepared for the test product and six for the control experiment.

The cells are brought into contact with the treatment medium for 72 h.

The incubation supernatants are removed for assay of the collagen VII. A protein assay is carried out on the tapetum of cells remaining in the wells (BCA method, SIGMA) for the purpose of relating the amounts of collagen VII to those of cellular proteins.

A plate treated in parallel is used for the XTT viability test with the XTT kit from Boehringer, reference 146 50 15, in order to measure the mitochondrial dehydrogenase activity of the viable cells. No significant drop in viability was detected by this test or by microscopic observation of the cells at the test concentration of 0.5 μg (microgram) per ml.

### d) ELISA of the Collagen VII

The protocol for assay of the collagen VII by an ELISA method was adapted from that used to assay collagen I (M. DUMAS, C. CHAUDAGNE, F. BONTE, A. MEYBECK: “In vitro biosynthesis of type I and III collagens by human dermal fibroblasts from donors of increasing age”, Mechanisms of Ageing and Development, 73 (1994) 179-187).

The following modifications were made:

1st antibody: mouse monoclonal antibody against human type VII collagen, IgG1 isotype (Life Technologies, ref. 12073-011, batch FB2b01).

2nd antibody: goat antibody against total mouse IgGs, coupled with alkaline phosphatase (Interchim, ref. 115-056-062, batch 26793).

The alkaline phosphatase activity is disclosed by cleavage of the substrate paranitrophenyl phosphate (PNPP) to paranitrophenol, whose absorbance is measured at 405 nanometers.

### e) Expression of the Results and Statistical Interpretation

The results of the increase in the proportion of collagen VII in the keratinocyte culture are expressed as the activity of the ellagic acid, denoted by A, where A is expressed as a percentage and corresponds to the following formula:

$$A = ( \frac{{RODC}_{ea} - {RODC}_{ks}}{{RODC}_{ks}} ) \times 100$$

$$where :$$

The subscript “ea” corresponds to the values associated with a keratinocyte culture prepared in the presence of ellagic acid.

The subscript “ks” corresponds to the values associated with a keratinocyte culture prepared in the absence of ellagic acid, which is also called a control culture.

ROD corresponds to the reduced optical density:

ROD=(OD−OD_{blank})

OD corresponds to an optical density measured following the treatment of a supernatant.

OD_{blank} corresponds to the optical density measured on the culture medium.

RODC corresponds to an ROD corrected so as to correspond to an optical density relating to 100 μg of cellular proteins assayed in the well in question.

The results obtained on the treated cultures (n=6) and control cultures (n=6) are compared by the unpaired Student test with a chosen significance level of p<0.05.

The activity of the product according to the invention, namely ellagic acid, on the proportion of collagen VII in the culture medium was thus tested on the two different strains of keratinocytes indicated above, referred to as NHK 1124 (P2) and NHK 1106 (P1).

### 2) Results—Conclusion

The results are given in Table 1 below on the basis of the mean of the measurements on the different cultures:

TABLE 1
ACTIVITY OF ELLAGIC ACID ON THE PROPORTION OF
COLLAGEN VII

| | | | | A for |
|---|---|---|---|---|
| | | | p (t test) | 72 h |
| | Ellagic acid | RODC for | relative to | of |
| Strain of | concentration | 72 h of | the control | incu- |
| keratinocytes | (μg/ml) | incubation | culture | bation |
| NHK 1124P2 | 0 | 0.773 ± 0.023 | | |
| (F, 49 years, | 0.5 | 1.098 ± 0.100 | 0.0003 | +42% |
| face-lift) | | | | |
| NHK 1106P1 | 0 | 0.950 ± 0.180 | | |
| (F, 50 years, | 0.5 | 1.563 ± 0.240 | 0.0007 | +64% |
| face-lift) | | | | |

The results given in Table 1 show a large and significant increase in the proportion of collagen VII present in the culture media due to the presence of the ellagic acid according to the invention at a concentration of 0.5 μg/ml.

As collagen VII is in particular the main constituent of the anchoring fibrils, it is clearly apparent from this test that ellagic acid and its derivatives according to the invention can advantageously be used as agents for reinforcing the dermal-epidermal junction and thereby improving the cohesion between the dermis and the epidermis. Ellagic acid and its derivatives according to the invention can therefore advantageously be used in cosmetic “antiwrinkle”, “anti-ageing” and “toning” compositions as well as in dermatological compositions for combating epidermolysis bullosa.

It is further known that collagen VII plays an important role at the level of the human hair follicle, which is why, given the experimental results above, there is great interest in ellagic acid and its derivatives according to the invention for hair care and, in general terms, for improving hair condition.

### Example 2 of the Invention

### Cosmetic Toning Composition

| | | |
|---|---|---|
| Ellagic acid commercially available from Extra Synthese, | 0.01 | g |
| France | | |
| Extract of Centella asiatica | 0.1 | g |
| Perfumed emulsified excipient in the form of an oil- | QSP 100 | g |
| in-water emulsion, and preservative | | |

This composition combats loosening of the skin and restores its firmness. It can advantageously be used for 3-week courses of treatment by application to the areas of the body to be treated where the skin is loose.

### Example 3 of the Invention

### “Anti-ageing”Emulsion

| | | |
|---|---|---|
| Acetate of ellagic acid | 0.02 | g |
| Vitamin A palmitate | 0.01 | g |
| Perfumed fluid emulsified excipient | QSP 100 | g |

This emulsion can be used on the areas of the body to be treated, particularly on the face, preferably every evening. It contributes towards delaying the appearance of signs of skin ageing, such as wrinkles or loosening of the skin. After daily treatment for about 6 months, the skin becomes smoother, more supple and firmer. It regains its radiance.

### Example 4 of the Invention

### Cosmetic Toning and “Anti-ageing” Emulsion

This emulsion is prepared from the following 3 phases A, B and C:

| Phase A | | |
|---|---|---|
| Emulgade SE ® (1) | 6 | g |
| Cetyl alcohol | 2 | g |
| BHT | 0.02 | g |
| Isononyl isononanoate | 6 | g |
| Propylparaben | 0.02 | g |
| Coconut glycerides | 6 | g |
| Centella asiatica | 0.1 | g |
| Phase B | | |
| Methylparaben | 0.2 | g |
| Distilled water | 67.64 | g |
| Phase C | | |
| Ellagic acid | 0.01 | g |
| Butylene glycol | 12 | g |
| Vitamin A palmitate | 0.01 | g |

(1) Emulgade SE® is a composition from HENKEL which is a mixture of glyceryl stearate (and) ceteareth-20 (and) ceteareth-10 (and) cetearyl alcohol (and) cetyl palmitate.

Phase C is prepared first by subjecting a mixture of the ellagic acid and the butylene glycol to an ultrasonic treatment for 15 minutes. The vitamin A palmitate is then added.

The fatty phase A and the aqueous phase B are heated together to 85° C. At 85° C., phase B is emulsified with phase A by mechanical agitation. Agitation is maintained while the resulting emulsion is cooled to 45° C., at which temperature agitation is continued for 45 minutes.

Phase C is added to the phase A/phase B emulsion, which is maintained at 45° C., with agitation. This agitation is continued for 5 minutes after phase C has been added.

This emulsion can be used on the areas of the body to be treated, particularly on the face, preferably every evening. It contributes towards delaying the appearance of signs of skin ageing, such as wrinkles, reducing the depth of the latter or combating loosening of the skin. After daily treatment for about six months, the skin becomes smoother, more supple and firmer.

### Example 5 of the Invention

### Cosmetic Toning and “Anti-ageing” Emulsion

This emulsion is prepared with the following compounds:

| | | |
|---|---|---|
| Distilled water | 67.53 | g |
| Butylene glycol | 12.00 | g |
| Emulgade SE ® | 6.00 | g |
| Isononyl isononanoate | 6.00 | g |
| Coconut glycerides | 6.00 | g |
| Cetyl alcohol | 2.00 | g |
| Methylparaben | 0.20 | g |
| Centella asiatica | 0.10 | g |
| Vitamin A palmitate | 0.10 | g |
| Sodium salt of ellagic acid | 0.03 | g |
| BHT | 0.02 | g |
| Propylparaben | 0.02 | g |

The composition Emulgade SE® is described in the previous Example.

This emulsion can be used on the areas of the body to be treated, particularly on the face, preferably every evening. It contributes towards delaying the appearance of signs of skin ageing, such as wrinkles, reducing the depth of the latter or combating loosening of the skin. After daily treatment for about six months, the skin becomes smoother, more supple and firmer.

### Example 6 of the Invention

### Dermatological Preparation in the Form of a Gel for the Treatment of Epidermolysis Bullosa

| | | |
|---|---|---|
| Zinc complex of ellagic acid | 0.01 | g |
| Butylene glycol | 10 | g |
| Absolute ethanol | 20 | g |
| Distilled water | 54.99 | g |
| Carbomer (Ultrez ®-10 gel from GOODRICH) at | 15 | g |
| a concentration of 2% | | |

This gel can be applied locally, three times a day for at least fifteen days, to the areas to be treated in cases of epidermolysis.

### Example 7 of the Invention

### “Antidandruff” Lotion for Combating Hair Loss

A hair lotion is prepared using 0.01 g of zinc complex of ellagic acid, 0.005 g of chloroxylenol and 0.01 g of cepharanthine with a perfumed alcoholic excipient to make up to 100 g.

The lotion is used by application to the scalp, morning and evening, followed by a gentle massage. After 8 to 15 days of treatment, hair loss has been distinctly slowed down and the itching sensation has disappeared. The recommended courses of treatment are for 30 days with intervals of 2 to 4 months, depending on the magnitude of the hair problem to be treated.

### Example 8 of the Invention

### Cosmetic Toning and “Anti-ageing” Emulsion

| | | |
|---|---|---|
| Ellagic acid | 0.50 | g |
| Vitamin A palmitate | 0.01 | g |
| Centella asiatica | 6.00 | g |
| Excipient with preservative | QSP 100.00 | g |

This emulsion can be used on the areas of the body to be treated, particularly on the face, preferably every evening. It contributes towards delaying the appearance of signs of skin ageing, such as wrinkles, reducing the depth of the latter or combating loosening of the skin. After daily treatment for about six months, the skin becomes smoother, more supple and firmer.

1. Use of ellagic acid, its salts, its metal complexes or its mono- or polyether or mono- or polyacylated derivatives as a cosmetic agent for improving the cohesion between the dermis and the epidermis, said agent being incorporated into a cosmetic composition comprising a cosmetically acceptable vehicle.

2. Use of ellagic acid, its salts, its metal complexes or its mono- or polyether or mono- or polyacylated derivatives as a cosmetic agent for increasing the proportion of collagen VII, said agent being incorporated into a cosmetic composition comprising a cosmetically acceptable vehicle.

3. Use according to claim 1 or 2, characterized in that said composition is intended for toning up the skin, preventing or delaying the appearance of signs of skin ageing, and delaying the appearance of wrinkles or reducing their depth.

4. Use according to claim 3, characterized in that the skin ageing is actinic ageing due in particular to the action of solar radiation.

5. Use according to claim 2, characterized in that said composition is a hair composition for improving hair condition.

6. Use of ellagic acid, its salts, its metal complexes or its mono- or polyether or mono- or polyacylated derivatives for the preparation of a pharmaceutical composition, especially dermatological composition, for treating pathological conditions associated with a deficiency in the cohesion between the dermis and the epidermis, particularly conditions associated with a weakening of the dermal-epidermal junction.

7. Use of ellagic acid, its salts, its metal complexes or its mono- or polyether or mono- or polyacylated derivatives for the preparation of a pharmaceutical composition, especially dermatological composition, for treating manifestations or pathological conditions associated with an insufficiency of collagen VII.

8. Use according to claim 6, characterized in that said composition is intended for treating epidermolysis bullosa.

9. Use according to one of claims 1 to 8, characterized in that the ellagic acid salts include metal salts, particularly those of alkali metals or alkaline earth metals such as sodium and calcium, amine salts such as those of methylglutamine, diethanolamine, triethanolamine, choline and bis-triethylamine, and amino acid salts, especially those of basic amino acids such as arginine, lysine and ornithine, the metal complexes of ellagic acid include those with zinc and copper, the mono- or polyacylated derivatives comprise saturated or unsaturated acyl groups having from 2 to 22 carbon atoms, these acyl groups preferably corresponding to acetic, palmitic, oleic, linoleic, linolenic, arachidonic, stearic, brassidic, erucic, behenic and (all Z)-5,8,11,14,17-eicosapentacnoic acids, and the above-mentioned mono- or polyether derivatives are derivatives of alkoxy comprising from 1 to 4 carbon atoms, or condensation derivatives of one or more of the hydroxyl groups of ellagic acid with a sugar or a chain of sugars, particularly 3-methoxyellagic acid or its mono- or polyether derivatives with sugars such as glucose, arabinose, rhamnose and galactose.

10. Use according to one of claims 1 to 9, characterized in that said composition contains from 0.0001% to 5% by weight, preferably between 0.01% and 1% by weight, of ellagic acid, its salts, its metal complexes or its mono- or polyether or mono- or polyacylated derivatives, based on the total weight of the composition.

11. Use according to one of claims 1 to 10, characterized in that said composition also contains at least one substance which favors the synthesis of the constituents of the extracellular matrix of the skin and/or regulates the formation of a good-quality corneal layer.

12. Use according to claim 11, characterized in that said substance is selected from the group consisting of vitamins, particularly vitamins of groups A and C, and their derivatives such as esters, tocopherols, xanthines, particularly caffeine or theophylline, retinoids, particularly vitamin A acid, extracts of Centella asiatica, asiatic and madecassic acids and their glycosylated derivatives such as asiaticoside or madecassoside, extracts of Siegesbeckia orientalis, extracts of Commiphora mukul and extracts of Eriobotrya japonica, and mineral elements.

13. Use according to claim 12, characterized in that the above-mentioned mineral elements are selected from magnesium, manganese, silicon and zinc, these mineral elements advantageously being used in the form of the chloride in the case of magnesium and manganese, in the form of silanol in the case of silicon and in the form of aspartate in the case of magnesium.

14. Use according to one of claims 1 to 13, characterized in that said composition also contains at least one substance selected from the group consisting of aliphatic C₃-C₁₂ alpha-hydroxy acids, particularly citric acid, malic acid and lactic acid, amino acids, particularly arginine, citrulline and threonine, ceramides, glycoceramides, phospholipids, slimming agents, particularly forskolin, extracts of Coleus, extracts of Tephrosia, agents for combating stretch marks, particularly extracts of horse-chestnut and escin, agents for protecting or improving the microcirculation, particularly the bioflavonoids of Ginkgo biloba, and sun filters, particularly titanium oxides, acyl methoxycinnamate and filters of vegetable origin.

15. Use according to one of claims 1 to 14, characterized in that said composition also contains at least one other active principle selected from the group consisting of antidandruff agents such as extracts of Arctium lappa, chloroxylenol, resorcinol or zinc pyrithione, antiseborrhea agents such as a 5α-reductase inhibitor, particularly an extract of Pygeum africanum, and agents for stimulating the blood microcirculation, such as cepharanthine and methyl nicotinate.

16. Method of cosmetic treatment for improving the cohesion between the dermis and the epidermis, particularly by reinforcing the dermal-epidermal junction, for toning up the skin, for preventing or delaying the appearance of signs of skin ageing, for delaying the appearance of wrinkles or reducing their depth, and for improving hair condition, characterized in that it consists in delivering a cosmetically effective amount of ellagic acid, its salts, its metal complexes or its mono- or polyether or mono- or polyacylated derivatives, optionally contained in a composition comprising a cosmetically acceptable excipient, particularly in a form as defined in one of claims 9 to 15.
